(19) United States
(12) Patent Application Publication
(10) Pub. No.: US 2026/0091204 A1
(43) Pub. Date: Apr. 2, 2026

(54) DEVICE TO ENSURE REMOVAL OF GUIDEWIRE A METHOD THEREOF

(71) Applicant: Vikas Raghove, Muncie, IN (US)

(72) Inventor: Vikas Raghove, Muncie, IN (US)

(21) Appl. No.: 18/904,267

(22) Filed: Oct. 2, 2024

(51) Int. Cl. A61M 25/09

(52) U.S. Cl. CPC A61M 25/09

(57) ABSTRACT

The present disclosure discloses a device and a method to ensure removal of guidewire from patient's body after catheterization. The device comprises a lid, a case, and a blocking pin. The lid and the case comprise one or more upper protrusions and one or more lower protrusions respectively, at a first side. The case comprises a first surface having a hole and a second surface having one or more holes for providing passage to one or more tubes of catheter. The blocking pin couples the one or more upper protrusions and the one or more lower protrusions at the first sides of both the lid and the case respectively to each other. Moreover, the blocking pin and a ridge-like structure at a second side of the case are configured to block the movement of the lid with respect to the case while the device is in a closed state.

### FIELD OF DISCLOSURE

The present disclosure is generally related to a device and a method to ensure removal of guidewire, and more particularly to a device and a method for ensuring safe removal of guidewire from patient's body after central venous catheterization.

### BACKGROUND

Various specialized medical techniques require proper training and expertise before implementing such treatments over patients. Medical personnel's for e.g., Doctors, Nurses, Surgeons etc. requires proper training for learning how to execute these specialized medical techniques in a safe and secure manner over patients. Moreover, there are a lot of medical procedures that becomes life-threatening due to minor mistakes of the doctor/surgeon. Catheterization is one such technique that requires extra precautions and care while being performed on the patient.

Catheterization is a process of insertion of a catheter inside body cavity of the patient that needs to be treated. Further, the catheterization is performed by the doctor/surgeon for the following purposes: 1. Allowing fluids to pass through. 2. Widening a passage of the body. Further, Catheterization are of three types: 1. Cardiac Catheterization, 2. Urinary Catheterization, 3. Central Venous Catheterization. The most sensitive processes of catheterizations are the cardiac catheterization and central venous catheterization. In both the process of catheterization, initial insertion of a guidewire is required. The guidewire is used for navigation, for placement of the catheter inside the body. Furthermore, the process involves insertion of some part of the catheter, (i.e., tubes) inside the body, and suturing of the remaining parts of the catheter (i.e., flange) on the body, over skin of the patient.

Moreover, during the process of catheterization, there are chances that the doctor/surgeon performing the procedure, by mistake fails to remove the guidewire before completing the procedure. Also, this leads to converting a simple bed-side procedure to a full-surgical operation. Indeed, this may cause on-the-spot fatalities. Hence, proper care should be taken while performing such procedures. Unfortunately, in present systems, there is no means to provide any reminders, for preventing such incidents.

In order to prevent guidewire retention and facilitate safe and effective catheterization, there is a need for an improved set of components that aids in safe and effective procedure. Therefore, there is a need for an improved device and method that may help in reducing and/or eliminating such fatalities and improving current scenario for both doctors and patients via safe removal of the guidewire from the patient's body before completing the catheterization with proper placement of the catheter.

### SUMMARY

A device to ensure removal of guidewire in accordance with the present disclosure comprises a lid, a case, and a blocking pin. The lid comprises one or more upper protrusions at a first side. The case comprises one or more lower protrusion at the first side. Further, the case comprises a first surface having a hole and a second surface having one or more holes for providing passage to one or more tubes. The blocking pin is configured to be coupled to the one or more upper protrusions and the one or more lower protrusions at the first sides of both the lid and the case respectively to each other. Further, the case comprises a ridge-like structure at a second side of the case for restricting movement of the lid with respect to the case at the second side. An engagement of the one or more upper protrusions of the lid and the one or more lower protrusions of the case via the blocking pin, and the restricted movement of the lid with respect to the case due to the ridge-like structure allows for the device to be in a closed state.

A method to ensure removal of guidewire from the patient's body after catheterization in accordance with the present disclosure, the method is being performed by the device comprising the lid, the case, and the blocking pin. The lid comprises the one or more upper protrusions at the first side. The case comprises the one or more lower protrusions at the first side, whereas the ridge-like structure at the second side. Further, the one or more upper protrusions and the one or more lower protrusions comprises the barrels. Also, the case is having the hole at the first surface and the one or more holes at the second surface. The hole on the first surface and the one or more holes on the second surface facilitates passage for the one or more tubes of the catheter. Furthermore, the blocking pin comprises the cylindrical rod having the first end and the second end. The blocking pin comprises the bulged surface near the first end of the blocking pin. The method comprises pulling by a surgeon/doctor, the guidewire after the catheterization, from the body of the patient that is being operated. The method further comprises inserting by the surgeon/doctor, one end of the guidewire into the barrels having the first end of the blocking pin of the device. The method includes ejecting via the guidewire, the second end of the blocking pin, from the barrels of the upper protrusions and the lower protrusions. The method comprises detaching by the surgeon/doctor, the lid, and the case from each other after the blocking pin is removed to thereby recover a flange. Moreover, the flange is configured to be secured within the device, and more specifically within a space formed by the lid and the case of the device. The method comprises suturing by the surgeon/doctor, the recovered flange with skin of the patient after the catheterization. Due to the bulged surface of the blocking pin, the blocking pin remains stuck at one of the barrel of either the upper protrusion of the lid or the lower protrusion of the case after detachment of the lid and the case.

In an embodiment of the present disclosure, the lid and the case are completely detachable from each other in an open state by removing the blocking from the device via inserting one end of the guidewire into barrels of either the one or more upper protrusions or the one or more lower protrusions that is having a first end of the blocking pin to eject the blocking pin from the barrel of either the upper protrusion or the lower protrusion having a second end of the blocking pin.

In an embodiment of the present disclosure, a shape of the device may be selected from any one of a cube and a cuboid.

In an embodiment of the present disclosure, the one or more upper protrusions of the lid, the one or more lower protrusions of the case and the blocking pin are of predetermined dimensions that are suitable for coupling the lid, the case, and the blocking pin together in the closed state.

In an embodiment of the present disclosure, the one or more upper protrusions, the one or more lower protrusions and the blocking pin are of predetermined dimensions that are suitable for coupling the lid, the case and the blocking pin together in the closed state. The predetermined dimensions of an inner diameter of barrels corresponding to the one or more upper protrusions and the one or more lower protrusions are wider than the predetermined dimensions of a diameter of the blocking pin, except a barrel that is narrower as compared to the bulged surface of the blocking pin. The predetermined dimensions allow for the secured accommodation of the blocking pin within the barrels of the one or more upper protrusions and the one or more lower protrusions, when the lid is covering the case such that the first sides and the second sides of each of the lid and the case are coinciding.

### BRIEF DESCRIPTION

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

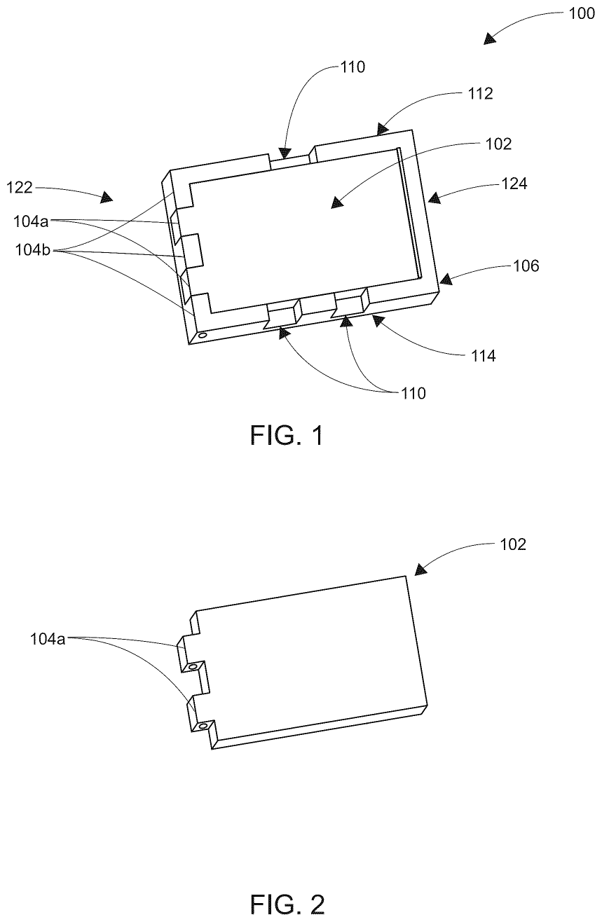

FIG. 1 illustrates an exemplary device to ensure removal of guidewire in accordance with the present disclosure.

FIG. 2 illustrates an exemplary lid of the device to ensure removal of the guidewire in accordance with the present disclosure.

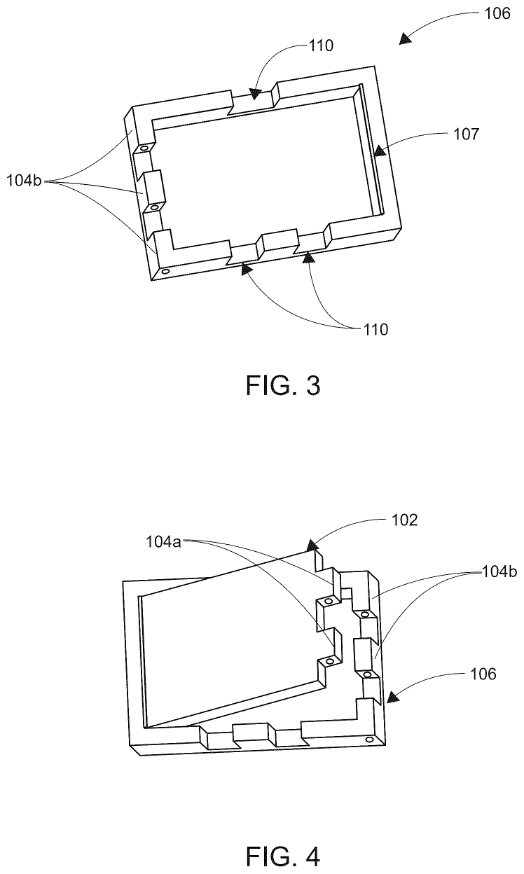

FIG. 3 illustrates an exemplary representation of a case of the device to ensure removal of the guidewire in accordance with the present disclosure.

FIG. 4 illustrates an exemplary representation of the device in an open state in accordance with the present disclosure.

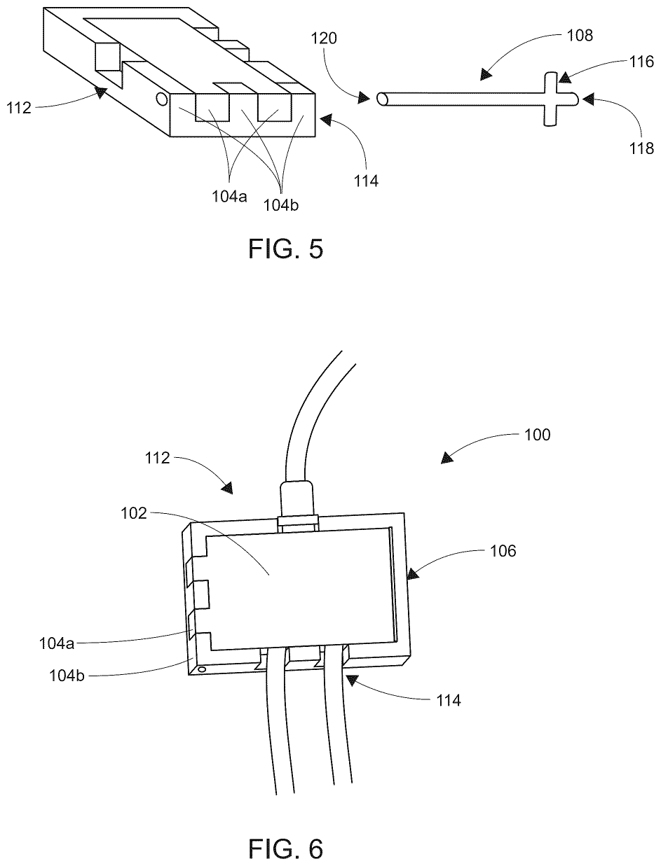

FIG. 5 illustrates an exemplary representation of a blocking pin and the case of the device in accordance with the present disclosure.

FIG. 6 illustrates an exemplary representation of the device encapsulating a catheter in accordance with the present disclosure.

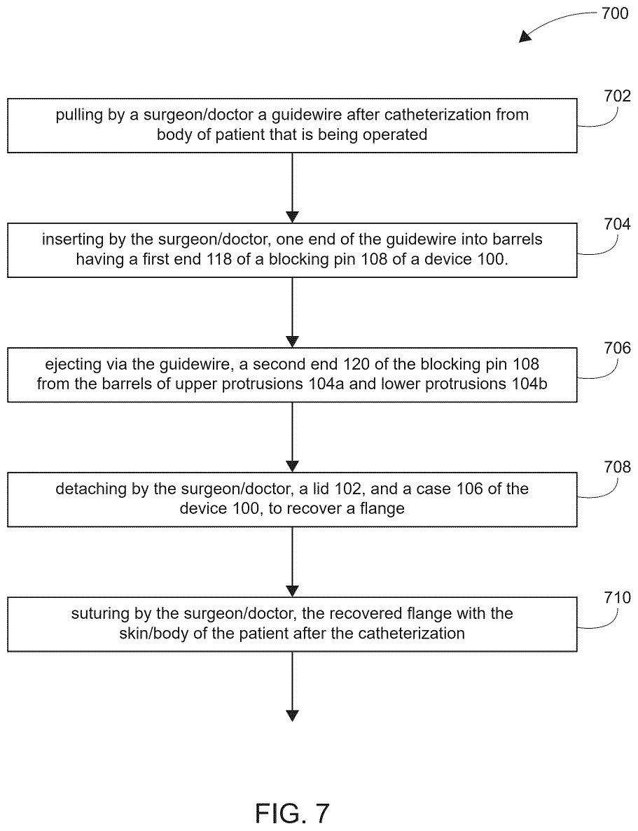

FIG. 7 illustrates an exemplary flowchart of a method to ensure removal of guidewire in accordance with the present disclosure.

### LIST OF REFERENCE NUMERALS

100—device
102—lid
104a—one or more upper protrusions
104b—one or more lower protrusions
106—case
107—ridge like structure
108—blocking pin
110—holes
112—first surface
114—second surface
116—bulged surface
118—first end
120—second end
122—first side
124—second side

### DETAILED DESCRIPTION

Embodiments of the present disclosure are best understood by reference to the figures and description set forth herein. All the aspects of the embodiments described herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit and scope thereof, and the embodiments herein include all such modifications.

As used herein, the term “exemplary” or “illustrative” means “serving as an example, instance, or illustration.” Any implementation described herein as exemplary or illustrative is not necessarily to be construed as advantageous and/or preferred over other embodiments. Unless the context requires otherwise, throughout the description and the claims, the word “comprise” and variations thereof, such as “comprises” and “comprising” are to be construed in an open, inclusive sense, i.e., as “including, but not limited to.”

This disclosure is generally drawn, inter alia, to methods, apparatuses, systems, devices implemented as tools for ensuring safe removal of guidewire from body cavity of patient after catheterization.

FIG. 1 illustrates an exemplary device 100 to ensure removal of guidewire from patient's body in accordance with the present disclosure. The device 100 is configured to ensure safe removal of the guidewire from the patient's body after catheterization via a catheter. The device 100 comprises a lid 102, a case 106, and a blocking pin 108. The lid 102 comprises one or more upper protrusions 104a at a first side 122 of the lid 102. The case 106 comprises one or more lower protrusions 104b at the first side 122 of the case 106. Each of the one or more upper protrusions 104a and the one or more lower protrusions 104b comprises barrels for allowing passage for passing the blocking pin 108 therewithin. A second side 124 of the case 106 comprises a ridge like structure 107. The ridge like structure 107 of the case 106 is configured to restrict movement of the lid 102 with respect to the case 106 at the second side 124, when the first sides 122 of each of the case 106 and the lid 102 are already coupled via the blocking pin 108. In an embodiment, the first side 122 and the second side 124 of the device 100 are opposite sides. In another embodiment, the first side 122 and the second side 124 of the device 100 are adjacent sides. Further, the case 106 comprises a first surface 112 having a hole 110 and a second surface 114 having one or more holes 110. The hole 110 at the first surface 112 of the case 106 and the one or more holes 110 at the second surface 114 of the case 106 are configured to provide passage for one or more tubes of the catheter. In an embodiment, the first surface 112 and the second surface 114 of the case 106 are on the opposite sides. In another embodiment, the first surface 112 and the second surface 114 of the case 106 are on the adjacent sides. The blocking pin 108 is configured to couple the one or more upper protrusions 104a of the lid 102 with the one or more lower protrusions 104b of the case 106, when the first sides 122 and the second sides 124 of each of the lid 102 and the case 106 are coinciding.

In an embodiment, the device 100 can be in a closed state or an open state. In an embodiment, when the device 100 is in the closed state, the device 100 is configured to enclose a flange of the catheter therewithin. When the device 100 is in the closed state, the first side 122 and the second side 124 of each of the lid 102 and the case 106 are coinciding, so as to carry the blocking pin 108 within the one or more upper protrusions 104a and the one or more lower protrusions 104b to couple the first side 122 of the lid 102 with the first side 122 of the case 106 and the movement of the second side 124 of the lid 102 is restricted with respect to the second side 124 of the case 106 due to the ridge like structure 107 of the case 106. Also, when the device 100 is in the closed state, the flange is carefully secured within the device 100 until the guidewire is removed. The flange is a portion of the catheter that is configured to be sutured to the patient's body after the catheterization. The flange can be taken out from the device 100 for suturing, by opening the device 100 via insertion of the guidewire into the device 100. To insert the guidewire into the device 100, the guidewire is first retrieved from the patient's body. Further, one end of the guidewire is inserted into the barrels of the device 100, thereby ejecting the blocking pin 108 from all except one of the barrel of either the upper protrusion 104a or the lower protrusion 104b of the device 100, leading to opening of the lid 102 of the device 100. In an embodiment, the device 100 may be of a shape selected from any one of a cube and a cuboid.

FIG. 2 illustrates an exemplary lid 102 of the device 100 to ensure removal of the guidewire in accordance with the present disclosure. The lid 102 comprises the one or more upper protrusions 104a at the first side 122. The lid 102 is configured to couple the case 106 of the device 100. When the device 100 is in the closed state, the first side 122 of the lid 102 having the one or more upper protrusions 104a, are coupled to the first side 122 of the case 106 having the one or more lower protrusions 104b and the second side 124 of the lid 102 is suppressed by the ridge like structure 107 at the second side 124 of the case 106.

FIG. 3 illustrates an exemplary representation of the case 106 of the device 100 to ensure removal of the guidewire in accordance with the present disclosure. The case 106 comprises the first side 122 and the second side 124. Further, the first side 122 of the case 106 comprises the one or more lower protrusions 104b and the second side 124 of the case 106 comprises the ridge like structure 107. The case 106 also comprises the first surface 112 and the second surface 114. The first surface 112 and the second surface 114 of the case 106 are on the opposite sides. The first surface 112 and the second surface 114 are not on a back side (not shown) of the case 106. In an embodiment, the first surface 112 and the second surface 114 of the case 106 can be either on the adjacent sides or on the opposite sides except the first side 122 and the back side of the case 106.

FIG. 4 illustrates an exemplary representation of the device 100 in the open state in accordance with the present disclosure. The device 100 is configured to be in the open state, when the lid 102 of the device 100 is detached from the case 106 via removal of the blocking pin 108. Further, the blocking pin 108 comprises a cylindrical rod having a first end 118 and a second end 120. The blocking pin 108 is removed from the device 100 via insertion of the one end of the guidewire into barrel of either the upper protrusion 104a or the lower protrusion 104b that is having the first end 118 of the blocking pin 108 to eject the blocking pin 108 from the barrel of either the upper protrusion 104a or the lower protrusion 104b having the second end 120 of the blocking pin 108. In the open state, the flange of the catheter is exposed and can be sutured to the body of the patient after the catheterization.

FIG. 5 illustrates an exemplary representation of the blocking pin 108 and the case 106 of the device 100 in accordance with the present disclosure. The blocking pin 108 comprises the cylindrical rod having the first end 118 and the second end 120. The blocking pin 108 comprises a bulged surface 116 near the first end 118 of the cylindrical rod. In an embodiment, the bulged surface 116 of the blocking pin 108 can be either a smooth surface or a rough surface. The blocking pin 108 is configured to be accommodated within the barrels of the one or more upper protrusions 104a and the one or more lower protrusions 104b of the device 100. Further, the one or more upper protrusions 104a of the lid 102, the one or more lower protrusions 104b of the case 106, and the blocking pin 108 are of predetermined dimensions that are suitable for coupling the lid 102, the case 106, and the blocking pin 108 together in the closed state. The predetermined dimensions of an inner diameter of barrels corresponding to the one or more upper protrusions 104a and the one or more lower protrusions 104b are wider than the predetermined dimensions of a diameter of the blocking pin 108, except a barrel that is narrower as compared to a bulged surface 116 of the blocking pin 108. The predetermined dimensions allow secured accommodation of the blocking pin 108 within the barrels of the one or more upper protrusions 104a and the one or more lower protrusions 104b when the lid 102 is covering the case 106 such that first sides 122 and second sides 124 of each of the lid 102 and the case 106 are coinciding. The accommodation of the blocking pin 108 within the barrels is such that the second end 120 of the blocking pin 108 is placed closer to the barrel of either the upper protrusion 104a or the lower protrusion 104b that is narrower as compared to other barrels, thereby providing narrower passage. The bulged surface 116 of the blocking pin 108 is configured to restrict the complete ejection of the blocking pin 108 or the first end 118 of the blocking pin 108 towards the barrel having narrower passage. The blocking pin 108 is inserted into the barrels of the one or more upper protrusions 104a and the one or more lower protrusions 104b from the second end 120 of the blocking pin 108 such that after insertion the second end 120 of the blocking pin 108 is closer to the barrel that is narrower as compared to the bulged surface 116 of the blocking pin 108.

In an embodiment, predetermined dimensions of the one or more upper protrusions 104a and the one or more lower protrusions 104b are different. In another embodiment, the predetermined dimensions of the one or more upper protrusions 104a and the one or more lower protrusions 104b are same except one barrel.

In an embodiment, the barrel that is providing narrower passage for the blocking pin 108 as compared to other barrels is either of the upper protrusion 104a or of the lower protrusion 104b of the device 100.

In an embodiment, each of the one or more upper protrusions 104a are having same predetermined dimensions. In another embodiment, each of the one or more upper protrusions 104a are having different predetermined dimension. In an embodiment, each of the one or more lower protrusions 104b are having same predetermined dimensions. In another embodiment, each of the one or more lower protrusions 104b are having different predetermined dimension.

FIG. 6 illustrates an exemplary representation of the device 100 encapsulating the catheter in accordance with the present disclosure. The device 100 is configured to encapsulate or enclose the flange of the catheter therewithin. Further, the one or more tubes of the catheter are penetrating from the one or more holes 110 on the device 100. The case 106 comprising the flange is tightly covered by the lid 102 of the device 100 using the blocking pin 108 and the ridge-like surface. The blocking pin 108 is coupled at the first side 122 of the device 100 at the one or more upper protrusions 104a and the one or more lower protrusions 104b. Further, the ridge-like structure is present at the second side 124 of the case 106 for blocking the movement of the lid 102 with respect to the case 106 when the first side 122 is having the blocking pin 108. The device 100 allows for careful catheterization and, also ensures safe removal of the guidewire before suturing the flange to the skin/body of the patient.

FIG. 7 illustrates an exemplary flowchart of a method 700 to ensure removal of guidewire from the patient's body after the catheterization in accordance with the present disclosure. The method 700 is configured to be performed by the device 100 comprising a lid 102, a case 106, and a blocking pin 108. The lid 102 of the device 100 comprises one or more upper protrusions 104a at the first side 122 of the device 100 and the case 106 comprises the one or more lower protrusions 104b at the first side 122 of the device 100. Further, the device 100 comprises the ridge like structure 107 at the second side 124 of the case 106. The one or more upper protrusions 104a and the one or more lower protrusions 104b comprises barrels. The case 106 comprises holes 110 at the first surface 112 and the one or more holes 110 at the second surface 114 of the case 106. The one or more holes 110 on the case 106 are configured to facilitate passage for the one or more tubes of the catheter. The blocking pin 108 comprises the cylindrical rod having the first end 118 and the second end 120. Also, the blocking pin 108 comprises the bulged surface 116 near the first end 118 of the blocking pin 108. The method 700 comprises the following steps:

In step 702, the method 700 comprises pulling by a surgeon/doctor the guidewire after the catheterization from the body of the patient that is being operated. The guidewire aids in placement of catheter in the body of the patient. After the placement of the catheter inside patient's body, the guidewire is removed by the surgeon/doctor. Also, the guidewire is utilized by the surgeon/doctor for performing further process after the catheterization.

In step 704, the method 700 comprises inserting by the surgeon/doctor, the one end of the guidewire into the barrels having the first end 118 of the blocking pin 108 of the device 100. The guidewire is inserted in the barrels of the one or more upper protrusions 104a and the one or more lower protrusions 104b of the lid 102 and the case 106 respectively. The barrels are having the blocking pin 108 therewithin. The guidewire is inserted in the barrel of either the upper protrusion 104a or the lower protrusion 104b from an end that aids in partial rather than complete removal of the blocking pin 108 from the barrels.

In step 706, the method 700 comprises ejecting via the guidewire, the second end 120 of the blocking pin 108 from the barrels of the upper protrusions 104a and the lower protrusions 104b. The continuous insertion of the guidewire inside the barrels from the first end 118 of the blocking pin 108 leads to ejection of the blocking pin 108 through the second end 120. The ejection of the blocking pin 108 through the second end 120 is partial but not complete, thereby preventing loss of the same in case 106 of complete ejection. Further, the partial ejection is beneficial as it prevents any further chances of fatalities caused by the unwanted loss of the blocking pin 108 in the patient body. Further, the partial ejection is due to the bulged surface 116 of the blocking pin 108 near the first end 118 as well as the narrower passage of the barrel near the second end 120 of the blocking pin 108. The bulged surface 116 prevents the blocking pin 108 to eject completely from the barrels of the device 100. The blocking pin 108 remains stuck at one of the barrel of either the upper protrusion 104a of lid 102 or the lower protrusion 104b of case 106.

In step 708, the method 700 comprises detaching by the surgeon/doctor, the lid 102, and the case 106, to recover the flange. As the blocking pin 108 is stuck at only one of the barrel of either the upper protrusion 104a or the lower protrusion, therefore allows for opening of the device 100 via the detachment of the lid 102 with respect to the case 106. As the device 100 is opened, the lid 102 and the case 106 are removed, and the flange is recovered from the device 100.

In step 710, the method 700 comprises suturing by the surgeon/doctor, the recovered flange with the skin/body of the patient after the catheterization. The recovery of the flange allows for careful suturing of the flange on the skin/body of the patient. After suturing, the flange is secured with the patient's body. Further, the use of the guidewire for recovery of the flange ensures the removal of guidewire from the body of the patient.

Accordingly, the device 100 of the present disclosure is advantageous in terms of providing a device 100 and a method 700 that ensures removal of the guidewire from the body of the patient after the catheterization. During catheterization, the catheter is to be threaded over the portion of the guidewire for placement inside the body vessels of the patient. Therefore, one end of the guidewire should be visible from an outside end of the catheter, when the catheter is placed in the body vessel. The guidewire grabbed from that end for enhancing the catheter inside the body of the patient to an appropriate position. Although, there are case 106s when the guidewire is completely lost with the placement of the catheter inside the body of the patient. This leads to a major surgery for recovering the guidewire than a simple bedside procedure of catheter placement. In conventional devices 100 and methods 700 there is no means for ensuring the careful removal of the guidewire before the flange is sutured to the patient body. Also, the presently existing devices 100 and methods 700 for catheterization do not provide any guarantee for the guidewire removal from the patient's body. Presently, there is a need of having such devices 100 and methods 700 that provides careful catheterization without any failure of leaving the guidewire inside the patient's body. Hence, the present disclosure is advantageous in terms of providing a device 100 and a method 700 that is efficient, accurate, safe, inexpensive, and less-time consuming and ensures removal of guidewire from the patient's body before catheterization. The device 100 comprises a lid 102 and a case 106 for enclosing the flange of the catheter therewithin. The only way to open the device 100 for suturing the flange on the body of the patient, is via the use of the guidewire that is inserted with the catheter in the body of the patient. Therefore, the guidewire is to be removed from the body of the patient after the placement of catheter, for opening the device 100 for recovering the flange.

Although the present disclosure has been described in terms of certain preferred embodiments, various features of separate embodiments can be combined to form additional embodiments not expressly described. Moreover, other embodiments apparent to those of ordinary skill in the art after reading this disclosure are also within the scope of this disclosure. Furthermore, not all the features, aspects and advantages are necessarily required to practice the present disclosure. Thus, while the above detailed description has shown, described, and pointed out novel features of the disclosure as applied to various embodiments, it will be understood that various omissions, substitutions, and changes in the form and details of the apparatus or process illustrated may be made by those of ordinary skill in the technology without departing from the spirit of the disclosure. The disclosures may be embodied in other specific forms not explicitly described herein. The embodiments described above are to be considered in all respects as illustrative only and not restrictive in any manner.

What is claimed is:

1. A device to ensure removal of guidewire from patient's body after catheterization, the device comprises:
a lid comprising one or more upper protrusions at a first side;
a case comprising one or more lower protrusions at the first side, the case comprises a first surface having a hole and a second surface having one or more holes for providing passage to one or more tubes; and
a blocking pin configured to couple the one or more upper protrusions and the one or more lower protrusions at the first sides of both the lid and the case respectively, to each other, wherein the case comprises a ridge like structure at a second side of the case for restricting movement of the lid with respect to the case at the second side, thereby blocking the movement of the lid with respect to the case while the device is in a closed state.

2. The device of claim 1, wherein in the closed state, the device is configured to enclose a flange of a catheter therewithin.

3. The device of claim 1, wherein the blocking pin comprises a cylindrical rod having a first end and a second end, the blocking pin comprises a bulged surface near the first end of the cylindrical rod.

4. The device of claim 1, wherein the lid and the case are completely detachable from each other in an open state by removing the blocking pin from the device via inserting one end of the guidewire into barrel of either the upper protrusion or the lower protrusion that is having a first end of the blocking pin to eject the blocking pin from the barrel of either the upper protrusion or the lower protrusion having a second end of the blocking pin.

5. The device of claim 1, wherein each of the one or more upper protrusions and the one or more lower protrusions comprises barrels for allowing passage for accommodating the blocking pin therewithin, the barrel formed by either the upper protrusion or the lower protrusion that is closer to a second end of the blocking pin provides a narrower passage as compared to other barrels, thereby restricting a first end of the blocking pin to eject out completely from that barrel due to a bulged surface of the blocking pin.

6. The device of claim 1, wherein the one or more upper protrusions of the lid, the one or more lower protrusions of the case, and the blocking pin are of predetermined dimensions that are suitable for coupling the lid, the case, and the blocking pin together in the closed state.

7. The device of claim 6, wherein the predetermined dimensions of an inner diameter of barrels corresponding to the one or more upper protrusions and the one or more lower protrusions are wider than the predetermined dimensions of a diameter of the blocking pin, except a barrel that is narrower as compared to a bulged surface of the blocking pin, such as to allow for secured accommodation of the blocking pin within the barrels of the one or more upper protrusions and the one or more lower protrusions, when the lid is covering the case such that first sides and second sides of each of the lid and the case are coinciding.

8. The device of claim 7, wherein the blocking pin is inserted from a second end of the blocking pin into the barrels of the one or more upper protrusions and the one or more lower protrusions such that after insertion the second end of the blocking pin is closer to the barrel that is narrower as compared to the bulged surface of the blocking pin.

9. The device of claim 1, wherein a shape of the device may be selected from any one of a cube and a cuboid.

10. A method to ensure removal of guidewire from patient's body after catheterization, the method is configured to be performed by a device comprising a lid, a case and a blocking pin, the lid comprises one or more upper protrusions at a first side, the case comprises one or more lower protrusions at the first side and a ridge like structure at a second side, the one or more upper protrusions and the one or more lower protrusions comprises barrels, the case is having a hole at a first surface and one or more holes at a second surface, wherein the hole and the one or more holes on the case are configured to facilitate passage for one or more tubes of a catheter, the blocking pin comprises a cylindrical rod having a first end and a second end, the blocking pin comprises a bulged surface near the first end, the method comprising:
pulling by a surgeon/doctor, the guidewire after the catheterization, from the body of the patient that is being operated;
inserting by the surgeon/doctor, one end of the guidewire into the barrels having the first end of the blocking pin of the device;
ejecting via the guidewire, the second end of the blocking pin, from the barrels of the upper protrusions and the lower protrusions;
detaching by the surgeon/doctor, the lid, and the case of the device, to recover a flange; and
suturing by the surgeon/doctor, the recovered flange with skin of the patient after the catheterization, wherein the blocking pin remains stuck at one of the barrel of either the upper protrusion or the lower protrusion after the detachment of the lid and the case, due to the bulged surface of the blocking pin.

11. The method of claim 10, wherein the barrels of each of the one or more upper protrusions and the one or more lower protrusions allows a passage for accommodating the blocking pin therewithin, the barrel formed by either the upper protrusion or the lower protrusion that is closer to the second end of the blocking pin provides narrower passage as compared to other barrels, thereby restricting the first end of the blocking pin to eject out completely from that barrel due to the bulged surface of the blocking pin.

12. The method of claim 10, wherein predetermined dimensions of an inner diameter of the barrels corresponding to the one or more upper protrusions and the one or more lower protrusions are wider than the predetermined dimensions for a diameter of the blocking pin, except a barrel that is narrower as compared to the bulged surface of the blocking pin, such as to allow for safe accommodation of the blocking pin within the barrels of the one or more upper protrusions and the one or more lower protrusions, when the lid is covering the case such that the first sides and the second sides of each of the lid and the case are coinciding.

13. The method of claim 10, wherein the one or more upper protrusions of the lid, the one or more lower protrusions of the case, and the blocking pin are of predetermined dimensions that are suitable for coupling the lid, the case, and the blocking pin together in a closed state.

14. The method of claim 10, wherein a shape of the device may be selected from any one of a cube and a cuboid.
